(19) United States
(12) Patent Application Publication
(10) Pub. No.: US 2018/0000718 A1
(43) Pub. Date: Jan. 4, 2018

(54) HAIR CLEANSING CONDITIONER

(71) Applicant: Henkel AG & Co. KGaA, Duesseldorf (DE)

(72) Inventors: Soeren Scheele, Hamburg (DE); Manuela Mette, Kleinfeld (DE); Thomas Schroeder, Hamburg (DE); Erik Schulze zur Wiesche, Hamburg (DE)

(73) Assignee: Henkel AG & Co. KGaA, Duesseldorf (DE)

(21) Appl. No.: 15/636,643

(22) Filed: Jun. 29, 2017

(30) Foreign Application Priority Data
Jun. 30, 2016 (DE) 10 2016 211 825.0

(51) Int. Cl. A61K 8/66; A61K 8/60; A61K 8/44; A61K 8/898

(52) U.S. Cl. CPC A61K 8/66; A61K 8/898; A61K 8/604; A61K 8/442; A61K 2800/5422; A61K 2800/5428

(57) ABSTRACT

Hair cleansing conditioners which include—in relation to the weight of the total composition—
a) from about 0.0001 to about 10 wt. % of a cationic 3-acrylamidopropyl trimethylammonium salt homopolymer, and
b) from about 0.01 to about 20 wt. % of at least one non-ionic surfactant and/or at least one amphoteric surfactant
   are exemplified by an excellent cleansing and conditioning effect. Hair treated with the hair cleansing conditioners has volume and fullness even after repeated use. There is no weighing-down of the hair (no build-up effect). In addition, treated hair has an improved feel and improved styling properties and improved combability. Such hair cleansing conditioners can also be easily rinsed out from the hair.

### CROSS-REFERENCE TO RELATED APPLICATION

This application claims priority to German Patent Application No. 10 2016 211 825.0, filed Jun. 30, 2016, which is incorporated herein by reference in its entirety.

### TECHNICAL FIELD

The present disclosure lies in the field of cosmetics and relates to hair cleansing conditioners which contain a cationic conditioning homopolymer and a non-ionic and/or an amphoteric surfactant.

### BACKGROUND

Conventional hair cleansers usually comprise anionic surfactants. These are desired and necessary so that sebum and other soiling can be removed from the hair surface and the scalp. However, they also result in a removal of lipids and proteins from the hair and/or the scalp during the cleaning process, and therefore a subsequent treatment with nourishing agents for regenerating the hair is necessary. Besides daily cleaning, however, the structure of hair can also be damaged by environmental influences (such as intense solar radiation), mechanical stresses (such as harsh combing under the heat of a hairdryer), and physical or chemical treatments (such as dyeing, shaping or smoothing the hair). The consequences are often split ends, hair breakage and/or a lack of hair shine. Hair damaged in this way is difficult to untangle and style. There is thus a need for particularly mild hair cleansers which degrease the hair as little as possible, even not at all, during the cleaning process and at the same time nourish the hair.

A new form of nourishing hair cleansers—what are known as hair cleansing conditioners—has been proposed in U.S. Pat. No. 6,723,309. These are based on a complex mixture of conditioning agents, cooling agents, emulsifiers, humectants, pH adjusters, preservatives, thickening agents, plant extracts, and vitamins. The hair cleansing conditioners cleanse the hair and provide it with greater shine, body, and improved styling properties. Disadvantages, however, of the hair cleansing conditioners include their low foaming capacity and their sometimes poor properties with regard to rinsing from the hair. In addition, thin and fine hair in particular has unsatisfactory hair volume following treatment with the hair cleansing conditioners, and repeated treatment with the hair cleansing conditioners can intensify the problem further still and can lead to an undesired and visually unappealing ‘weighing-down’ of the hair (what is known as a ‘build-up’ effect).

### BRIEF SUMMARY

Hair cleansing conditioners and methods for treating hair are provided herein. In an embodiment, a hair cleansing conditioner includes, in relation to the weight of the total composition,

a) from about 0.0001 to about 10 wt. % of a cationic 3-acrylamidopropyl trimethylammonium salt homopolymer, and
b) from about 0.01 to about 20 wt. % of at least one non-ionic surfactant and/or at least one amphoteric surfactant.

In another embodiment, a method for treating hair is provided. In accordance with the method, a hair cleansing conditioner is applied to the hair, is massaged in, and is rinsed out after a reaction time. The hair cleansing conditioner includes, in relation to the weight of the total composition,

a) from about 0.0001 to about 10 wt. % of a cationic 3-acrylamidopropyl trimethylammonium salt homopolymer, and
b) from about 0.01 to about 20 wt. % of at least one non-ionic surfactant and/or at least one amphoteric surfactant.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure is therefore to provide hair cleansing conditioners which do not have the above-mentioned disadvantages. In particular, hair cleansing conditioners that have good foaming capability should be provided, which ideally did not contain any anionic (sulphate) surfactants and which ensure a particularly mild cleansing. At the same time, hair of any type (but in particular brittle, damaged, dull, thin and/or lank hair) should be effectively nourished so that it is softer and has greater volume, elasticity and shine following the treatment with the hair cleansing conditioners.

It has now surprisingly been found that the aforementioned objects are achieved outstandingly by hair cleansing conditioners which, besides a specific cationic homopolymer, also contain at least one non-ionic and/or at least one amphoteric surfactant.

Such hair cleansing conditioners lead to products that cleanse effectively and gently with a very good hair conditioning effect, which provide the hair with fullness even with repeated use, and which do not weigh down the hair (no build-up effect). In addition, treated hair has an improved feel and also improved styling properties and improved combability.

In addition, hair cleansing conditioners of this type can be rinsed out very well from the hair.

A first subject of this application is a hair cleansing conditioner which contains—in relation to the weight of the total composition—

a) from about 0.0001 to about 10 wt. % of a cationic 3-acrylamidopropyl trimethylammonium salt homopolymer, and
b) from about 0.01 to about 20 wt. % of at least one non-ionic surfactant and/or at least one amphoteric surfactant.

The hair cleansing conditioners as contemplated herein contain the active substances a) and b) preferably in a suitable cosmetic carrier. This is understood within the scope as contemplated herein to mean preferably an aqueous carrier which contains (in relation to the total weight of the hair cleansing conditioners) preferably at least about 60 wt. %, more preferably at least about 65 wt. %, particularly preferably at least about 70 wt. %, and in particular preferably at least about 75 wt. %, of water. The cosmetic carrier can also contain from about 0.01 to about 30 wt. %, preferably from about 0.05 to about 20 wt. %, and in particular from about 0.1 to about 10 wt. %, of at least one alcohol. Suitable alcohols are, for example, ethanol, ethyldiglycol, 1-propanol, 2-propanol, isopropanol, 1,2-propylene glycol, 1,3-propylene glycol, 1-butanol, 2-butanol, 1,2-butanediol, 1,3-butanediol, 1,4-butanediol, 1-pentanol, 2-pentanol, 1,2-pentanediol, 1,5-pentanediol, 1-hexanol, 2-hexanol, 1,2-hexanediol, 1,6-hexanediol, glycerol, polyethylene glycol, sorbitol, sorbitan, benzyl alcohol, phenoxyethanol or mixtures of these alcohols. The water-soluble alcohols are preferred, such as ethanol, 1,2-propylene glycol, glycerol, sorbitol, benzyl alcohol and/or phenoxyethanol and mixtures of these alcohols. Glycerol and/or 1,2-propylene glycol are preferred in particular.

The hair cleansing conditioners as contemplated herein contain, as constituent a), a cationic 3-acrylamidopropyl trimethylammonium salt homopolymer, preferably a (3-acrylamidopropyl)trimethylammonium chloride homopolymer. Such polymers have a high cationic charge density and therefore bind effectively and completely to the negative charges of human hair. Cationic (3-acrylamidopropyl)trimethylammonium chloride homopolymers which have a mean molecular mass Mr of approximately 300,000 are preferred.

In a preferred embodiment the hair cleansing conditioners as contemplated herein contain a (3-acrylamidopropyl)trimethylammonium chloride homopolymer which is commercially available under the INCI name Polyacrylamidopropyltrimonium Chloride. In a very particularly preferred embodiment the hair cleansing conditioners as contemplated herein contain a (3-acrylamidopropyl)trimethylammonium chloride homopolymer which is obtainable from the company Ashland under the trade name N-DurHance A-1000®.

The hair cleansing conditioners as contemplated herein contain the cationic 3-acrylamidopropyltrimethylammonium salt homopolymer a) in an amount of from about 0.0001 to about 10 wt. %, in relation to the weight of the hair cleansing conditioners. Used amounts of the cationic homopolymer a) of from about 0.001 to about 5.0 wt. %, particularly preferably from about 0.005 to about 3.0 wt. %, and in particular from about 0.01 to about 2.0 wt. % are preferred.

A second essential ingredient of the hair cleansing conditioners as contemplated herein is at least one non-ionic and/or at least one amphoteric surfactant b). A content of actively cleansing—preferably mild—surfactants b) is necessary in order to attain a sufficient foaming capacity of the hair cleansing conditioners as contemplated herein.

In a first particularly preferred embodiment the hair cleansing conditioners as contemplated herein contain at least one non-ionic surfactant (bi).

Suitable non-ionic surfactants b) in the sense of the present disclosure can preferably be selected from

addition products of from 4 to about 30 mol ethylene oxide and/or from 0 to 5 mol propylene oxide with linear fatty alcohols having 8 to 22 C atoms, with fatty acids having 12 to 22 C atoms, and with alkylphenols having 8 to 15 C atoms in the alkyl group,

ethylene oxide and polyglycerol addition products with methyl glucoside fatty acid esters and fatty acid glucamides,

C8-C30 fatty acid mono- and diesters of addition products of from 1 to about 30 mol ethylene oxide with glycerol,

amine oxides,

sugar fatty acid esters and addition products of ethylene oxide with sugar fatty acid esters and/or

alkyl(oligo)glycosides,

mixtures of alkyl(oligo)glucosides and fatty alcohols, for example the commercially available product Montanov-68,

addition products of from 5 to about 60 mol ethylene oxide with castor oil and hardened castor oil,

partial esters of polyols having 3-6 carbon atoms with saturated fatty acids having from 8 to 22 C atoms,

sterols. Sterols are understood to mean a group of steroids which carry a hydroxyl group at C atom 3 of the steroid structure and are isolated both from animal tissue (zoosterols) and from plant fats (phytosterols). Examples of zoosterols are cholesterol and lanosterol. Examples of suitable phytosterols are ergosterol, stigmasterol and sitosterol. Sterols are also isolated from fungi and yeasts and in this case are known as mycosterols.

Phospholipids. These include in particular the glucose phospholipids, which for example are obtained as lecithins or phosphatidylcholines from, for example, egg yolk or plant seeds (for example soya beans).

Particularly suitable alkyl(oligo)glycosides are selected from compounds of general formula RO-[G]x, in which [G] preferably derives from aldoses and/or ketoses having 5-6 carbon atoms, preferably from glucose. The index number x stands for the degree of oligomerisation (DP), i.e. for the distribution of the monoglycosides and oligoglycosides. The index number x preferably has a value in the range from about 1 to about 10, particularly preferably in the range from about 1 to about 3, wherein it is not an integer, but can be a fractional number, which can be determined analytically. Particularly preferred alkyl(oligo)glycosides have a degree of oligomerisation between about 1.2 and about 1.5. The group R stands preferably for at least one alkyl and/or alkenyl group having 4 to 24 C atoms. Alkyl(oligo)glycosides that are preferred in particular are the compounds known under the INCI names Caprylyl/Capryl Glucoside, Decyl Glucoside, Lauryl Glucoside and Coco Glucoside.

Particularly suitable amine oxides are selected from at least one compound of general formula (I) or (II)

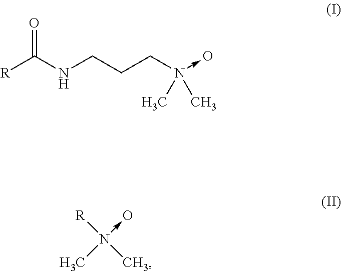

in which R stands in each case for a straight-chained or branched, saturated or mono- or polyunsaturated alkyl group or alkenyl group having 6 to 24 carbon atoms, preferably having 8 to 18 carbon atoms. The surfactants of the aforementioned formula (I) or (II) obtainable under the INCI names Cocamine Oxide, Lauramine Oxide and/or Cocamidopropylamine Oxide and commercially obtainable from various suppliers are preferred in particular.

Suitable C8-C30 fatty acid mono- and diesters of addition products of from 1 to 30 mol ethylene oxide with glycerol are preferably understood to be those known under the INCI names PEG(1-10) Glyceryl Cocoate, in particular PEG-7 Glyceryl Cocoate. It can also be advantageous to combine the ethoxylated glyceryl esters with further ethoxylated glyceryl esters. Such product mixtures are commercially obtainable—for example under the name “Antil 200®” (INCI name: PEG-200 Hydrogenated Glyceryl Palmate, PEG-7 Glyceryl Cocoate) from the company Evonik.

Mild non-ionic surfactants (bi) having a high foaming power which advantageously can be combined particularly well with cationic compounds a), in addition to further cationic compounds, to form stable compositions which are readily distributed over and rinsed out from the hair are used in the hair cleansing conditioners as contemplated herein with particular preference.

In the sense of the present disclosure, the mild surfactants (bi) having a high foaming power and which therefore are particularly preferably non-ionic surfactants include the aforementioned alkyl(oligo)glycosides and amine oxides. Alkyl(oligo)glycosides are preferred in particular.

In a preferred embodiment the hair cleansing conditioners as contemplated herein therefore contain, as non-ionic surfactant (bi), an alkyl(oligo)glycoside of general formula RO-[G]x, in which R stands for an alkyl group and/or alkenyl group having 4 to 22 C atoms, G stands for a sugar group having 5 to 6 C atoms, and x stands for the numbers from 1 to 10. Within this embodiment it is particularly preferred if the non-ionic surfactant (bi) is selected from at least one of the compounds known under the INCI names Caprylyl/Capryl Glucoside, Decyl Glucoside, Lauryl Glucoside and Coco Glucoside.

The non-ionic surfactant(s) can be used in the hair cleansing conditioners as contemplated herein preferably in a proportion by weight of from about 0.01 to about 10 wt. %, more preferably from about 0.10 to about 7.50 wt. %, particularly preferably from about 0.50 to about 6.00 wt. %, and in particular from about 1.00 to about 5.00 wt. %, in the total weight of the hair cleansing conditions. The hair cleansing conditioners as contemplated herein particularly preferably contain alkyl(oligo)glycosides in the aforementioned amounts.

A further preferred embodiment as contemplated herein is therefore exemplified in that the hair cleansing conditioners—in relation to their weight—contains from about 0.01 to about 10 wt. %, more preferably from about 0.10 to about 7.50 wt. %, particularly preferably from about 0.50 to about 6.00 wt. %, and in particular from about 1.00 to about 5.00 wt. %, of at least one non-ionic surfactant (bi), preferably an alkyl(oligo)glycoside, and in particular preferably at least one of the compounds known under the INCI names Caprylyl/Capryl Glucoside, Decyl Glucoside, Lauryl Glucoside and/or Coco Glucoside.

For some embodiments, it can be advantageous if the hair cleansing conditioners as contemplated herein also contain, besides the at least one non-ionic surfactant (bi) and/or the amphoteric surfactant (bii), at least one further non-ionic surfactant (bii)—different from (bi) which is suitable for stabilising the foam of the compositions as contemplated herein and for providing it with a pleasant feel. Particularly preferred non-ionic surfactants (biii) are what are known as the fatty acid alkanolamides of the following general formula (III),

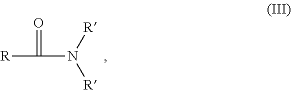

in which R preferably means a linear or branched, saturated or unsaturated alkyl group or alkenyl group having 8 to 24 carbon atoms, and the groups R′ stand for hydrogen or for the group —(CH2)nOH, in which n means the numbers 2 or 3, with the provision that at least one of the groups R′ stands for the aforementioned group —(CH2)nOH. The compounds known under the INCI names Cocamide MEA, Cocamide MIPA and/or Cocamide DEA, particularly Cocamide MEA, which are commercially obtainable from various providers, for example from the company BASF under the trade name Comperlan®100, are preferred in particular.

In a further preferred embodiment the hair cleansing conditioners as contemplated herein contain—in relation to their weight—additionally from about 0.10 to about 10.00 wt. %, more preferably from about 0.25 to about 7.50 wt. %, particularly preferably from about 0.50 to about 5.00 wt. %, and in particular from about 0.75 to about 4.00 wt. % of at least one fatty acid alkanolamide of the aforementioned formula (III), preferably a compound known under the INCI name Cocamide MEA.

In a second particularly preferred embodiment the hair cleansing conditioners as contemplated herein contain at least one amphoteric surfactant (bii). The term “amphoteric” surfactant is understood as contemplated herein to mean amphoteric and/or zwitterionic surfactants.

Suitable amphoteric and/or zwitterionic surfactants (bii) in the sense of the present disclosure can be selected preferably from one or more compounds of the following formulas (IV) to (X), in which the group R stands in each case for a straight-chained or branched, saturated or mono- or polyunsaturated alkyl group or alkenyl group having 7 to 23 carbon atoms (formulas (IV) and (V)) or for a straight-chained or branched, saturated or mono- or polyunsaturated alkyl group or alkenyl group having 8 to 24 carbon atoms (formulas (VI) to (X)):

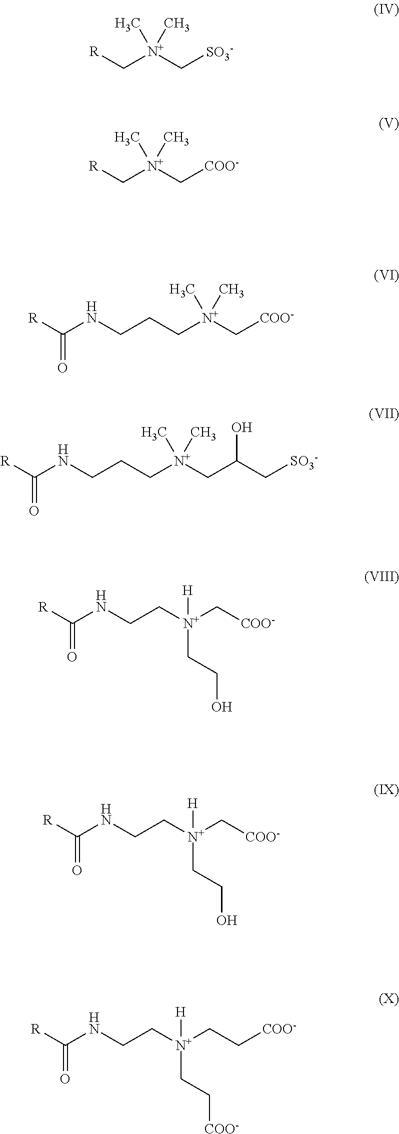

Preferred amphoteric and/or zwitterionic surfactants of one of the aforementioned formulas (IV) to (X) contain, as group R, predominantly a straight-chained or branched, saturated, mono- or polyunsaturated alkyl group having 8 to 20, more preferably from 8 to 18, and in particular having 8 to 16 C atoms. Particularly preferred are amphoteric and/or zwitterionic surfactants in which the group R derives from coconut fat. The amphoteric/zwitterionic surfactants known under the INCI names Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaine, Lauryl Betaine, Cocamidopropyl Betaine and/or Lauramidopropyl Betaine and commercially available from a number of suppliers are very particularly preferred. Surfactants having the INCI names Cocamidopropyl Betaine and/or Cocoampho(di)acetate are preferred in particular.

The proportion by weight of the at least one amphoteric surfactant (bii) in the total weight of the hair cleansing conditioners as contemplated herein is preferably from about 0.10 to about 15.00 wt. %, preferably from about 0.50 to about 12.50 wt. %, particularly preferably from about 1.00 to about 10.00 wt. %, and in particular from about 1.50 to about 7.50 wt. %. The hair cleansing conditioners as contemplated herein particularly preferably contain amphoteric surfactants (bii) known under the INCI names Cocamidopropyl Betaine and/or Cocoampho(di)acetate in the aforementioned amounts.

A further preferred embodiment as contemplated herein is exemplified in that the hair cleansing conditioners contain from about 0.10 to about 15.00 wt. %, preferably from about 0.50 to about 12.50 wt. %, particularly preferably from about 1.00 to about 10.00 wt. %, and in particular from about 1.50 to about 7.50 wt. %, of at least one amphoteric surfactant (bii), preferably one of the compounds known under the INCI names Sodium Cocoampho(di)acetate and/or Cocamidopropyl Betaine.

The hair cleansing conditioners as contemplated herein demonstrate an excellent nourishing and/or regreasing capacity in addition to excellent foaming properties, without any anionic surfactants having to be added to them.

In addition, they have excellent mildness and are particularly suitable for the treatment of damaged hair and/or a sensitive, dry scalp.

In a further preferred embodiment, the hair cleansing conditioners as contemplated herein are therefore substantially free from anionic surfactants.

Within this embodiment, it is particularly preferred if the hair cleansing conditioners as contemplated herein are substantially free from anionic sulphate surfactants.

The term “substantially free from” is understood to mean preferably a content of anionic (sulphate) surfactants of at most about 0.50 wt. %, more preferably of at most about 0.30 wt. %, particularly preferably of at most about 0.10 wt. %, and in particular of 0 wt. %, wherein the specified amounts relate to the total weight of the hair cleansing conditioners as contemplated herein. The specified maximum amounts apply for anionic (sulphate) surfactants which can be added freely and/or as aqueous solutions to the hair cleansing conditioners as contemplated herein, and not for hypothetical amounts which can be contained in various commercial products in minor amounts as minor constituent.

The hair cleansing conditioners as contemplated herein are preferably conventional rinse-off products, such as a nourishing shampoo and/or a foaming nourishing conditioner that can be rinsed out. It is also possible and preferred for some application forms, however, if the hair cleansing conditioners as contemplated herein are applied as a cleansing hair mousse. A particularly good distribution and dosing of the hair cleansing conditioners is ensured as a result.

The hair cleansing conditioners as contemplated herein are therefore present in the form of a cleansing nourishing mousse in accordance with a further particularly preferred embodiment. For this purpose, the hair cleansing conditioners as contemplated herein are provided in a dispensing device which is either a compressed gas container (aerosol container) filled additionally with a propellant, or is a non-aerosol container. The compressed gas container, with the aid of which a product is dispensed via a valve by employing the internal gas pressure of the container, is referred to as an “aerosol container” by definition. By contrast to the aerosol definition, a “non-aerosol container” is defined as a container under normal pressure, with the aid of which a product is dispensed by a pump or squeezing system by employing mechanical action. Within this embodiment, the hair cleansing conditioners are particularly preferably present in the form of an aerosol mousse in an aerosol container. The hair cleansing conditioners therefore preferably additionally contain at least one propellant. Hair cleansing conditioners as contemplated herein which are present in the form of an aerosol product can be produced in the conventional way. All constituents of the hair cleansing conditioners as contemplated herein with the exception of the propellant are generally filled into a suitable pressure-tight container. This container is then closed by employing a valve. The desired amount of propellant is lastly added by employing conventional techniques. In the embodiment in the form of an aerosol mousse, propellants that are suitable as contemplated herein are selected by way of example from N2O, dimethyl ether, CO2, air, alkanes having 3 to 5 carbon atoms, such as propane, n-butane, iso-butane, n-pentane and iso-pentane, and mixtures thereof. In accordance with the embodiment of an aerosol mousse, the aforementioned alkanes, mixtures of the aforementioned alkanes, or mixtures of the aforementioned alkanes with dimethyl ether are used as sole propellant. However, the present disclosure also expressly includes the co-use of propellants of the chlorofluorocarbon type, but in particular the fluorinated hydrocarbons. Dimethyl ether, propane, n-butane, iso-butane and mixtures thereof are preferred. Mixtures of propane and butane are very particularly preferably used as sole propellant in a proportion by weight of propane to butane of from about 70 to about 30 up to from about 15 to about 85. These mixtures are in turn preferably used in the cosmetic hair cleansing conditioners as contemplated herein in an amount of from about 1.00 to about 50.0 wt. %—in relation to the total weight of the total hair cleansing conditioners. Amounts of from about 2.00 to about 40.0 wt. % are more preferred, particularly preferably from about 3.00 to about 30.0 wt. %, and in particular from about 4.00 to about 20.0 wt. %. As contemplated herein, butane is understood to mean n-butane, iso-butane, and mixtures of n-butane and iso-butane.

In a third particularly preferred embodiment, the hair cleansing conditioner as contemplated herein is present in the form of an aerosol mousse in an aerosol container and additionally contains at least one propellant. Within the third particularly preferred embodiment, it is particularly advantageous if the hair cleansing conditioners as contemplated herein contain the propellant—preferably a mixture of propane/butane—in a proportion by weight of from about 1.00 to about 50.0 wt. %, preferably from about 2.00 to about 40.0 wt. %, particularly preferably from about 3.00 to about 30.0 wt. %, and in particular from about 4.00 to about 20.0 wt. %, in the total weight of the composition.

Besides good foaming properties and a particularly mild cleansing action, the hair cleansing conditioners as contemplated herein should additionally have an excellent nourishing effect and—in particular in the case of damaged hair—should restore shine, elasticity and volume.

It has been found that the nourishing properties of the agents as contemplated herein can be increased further still if, besides the cationic homopolymer a), at least one further hair-conditioning active substance c) is added to said agents. Protein hydrolysates have proven to be particularly suitable additional nourishing active substances c) because they increase, in combination with the cationic homopolymer a), not only the shine, the elasticity and/or the volume of the hair, but in addition improve the structure in particular of previously damaged hair and make it smoother and softer, with a nice feel.

In a fourth particularly preferred embodiment, the hair cleansing conditioners as contemplated herein therefore additionally contain at least one protein hydrolysate c).

Suitable protein hydrolysates c) in the sense of the present disclosure are understood to mean product mixtures which can be obtained by acid-, base- or enzyme-catalysed degradation of proteins (albumens). Protein hydrolysates of plant, animal and/or marine origin can be used.

Animal protein hydrolysates are, for example, elastin, collagen, keratin, silk and lactoprotein protein hydrolysates, which can also be present in the form of salts. Such products are marketed for example under the trade names Dehylan® (Cognis), Promois® (Interorgana) Collapuron® (Cognis), Nutrilan® (Cognis), Gelita-Sol® (German gelatin factories Stoess & Co), Lexein® (Inolex), proSina® (Croda) and Kerasol® (Croda).

Suitable protein hydrolysates of plant origin are, for example, soy, almond, rice, pea, potato and wheat protein hydrolysates. Such products are obtainable, for example, under the trade names Gluadin® (Cognis), DiaMin® (Diamalt), Lexein® (Inolex) and Crotein® (Croda).

Cationised protein hydrolysates can also be used, wherein the basic protein hydrolysate can originate from animals, for example from collagen, milk or keratin, from plants, for example from wheat, maize, rice, potatoes, soya or almonds, from marine life forms, for example from fish collagen or algae, or from biotechnologically obtained protein hydrolysates. The protein hydrolysates forming the basis of the cationic derivatives can be obtained from the corresponding proteins by a chemical hydrolysis, particularly alkaline or acid hydrolysis, by an enzymatic hydrolysis and/or a combination of both types of hydrolysis. The hydrolysis of proteins generally produces a protein hydrolysate with a molecular weight distribution from about 100 daltons up to several thousand daltons. Cationic protein hydrolysates that are preferred are those of which the base protein content has a molecular weight of from about 100 to about 25,000 daltons, preferably from about 250 to about 5,000 daltons. Moreover, cationic protein hydrolysates are understood to include quaternised amino acids and their mixtures. Quaternisation of the protein hydrolysates or the amino acids is often carried out using quaternary ammonium salts, such as N,N-dimethyl-N-(n-alkyl)-N-(2-hydroxy-3-chloro-n-propyl) ammonium halides. Moreover, the cationic protein hydrolysates can also be further derivatised. Typical examples of cationic protein hydrolysates and derivatives are the commercially available products known under the INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein.

Particularly good nourishing results can be attained if the hair cleansing conditioners as contemplated herein contain, as protein hydrolysate c), at least one protein hydrolysate of animal origin, preferably a (possibly cationically modified) silk and/or keratin hydrolysate and in particular a (possibly cationically modified) keratin hydrolysate, for example one of the products obtainable from the company Croda under the trade names ProSina® and/or Croquat® WKP PE LQ.

The proportion by weight of the protein hydrolysate(s) c) in the total weight of the hair cleansing conditioners as contemplated herein is preferably from about 0.01 to about 5.00 wt. %, more preferably from about 0.05 to about 4.00 wt. %, particularly preferably from about 0.10 to about 3.00 wt. %, and in particular from about 0.10 to about 2.00 wt. %.

In a fifth particularly preferred embodiment, the hair cleansing conditioners as contemplated herein contain—in relation to their total weight—

a) from about 0.0001 to about 10 wt. % of a cationic 3-acrylamidopropyl trimethylammonium salt homopolymer, preferably a (3-acrylamidopropyl)trimethylammonium chloride homopolymer,
b) from about 0.01 to about 10 wt. % of at least one non-ionic surfactant (bi), preferably an alkyl(oligo)glycoside, and
c) from about 1.00 to about 50.0 wt. % of at least one propellant, preferably a mixture of propane and butane, and/or
from about 0.01 to about 5.00 wt. % of at least one protein hydrolysate, preferably a protein hydrolysate known under the INCI name Hydrolyzed Keratin.

In a sixth particularly preferred embodiment, the hair cleansing conditioners as contemplated herein contain—in relation to their total weight—
a) from about 0.0001 to about 10 wt. % of a cationic 3-acrylamidopropyl trimethylammonium salt homopolymer, preferably a (3-acrylamidopropyl)trimethylammonium chloride homopolymer,
b) from about 0.10 to about 15 wt. % of at least one amphoteric surfactant (bii), preferably a compound known under the INCI name Cocoampho(di)acetate and/or Cocamidopropyl Betaine, and
c) from about 1.00 to about 50 wt. % of at least one propellant, preferably a mixture of propane and butane, and/or
   from about 0.01 to about 5.00 wt. % of at least one protein hydrolysate, preferably a protein hydrolysate known under the INCI name Hydrolyzed Keratin.

In order to further increase the nourishing properties, the hair cleansing conditioners as contemplated herein can contain yet further hair-conditioning active substances d). Besides an excellent nourishing effect, the further hair-conditioning active substances d) must be able to be easily incorporated into the hair cleansing conditioners and should not negatively influence the stability and/or foaming properties thereof. Care should also be taken to ensure that the combination of the further nourishing substances with the cationic nourishing polymer a) and, where applicable, with the protein hydrolysate(s) c) does not result in an overconditioning of the hair, which can lead to an undesired weighing-down of the hair and—with regular use—to an undesirable ‘build-up’ effect.

Thus,

cationic surfactants (di),

vitamins (dii) and/or

oil, fat and/or wax components (diii)

have proven to be particularly advantageous further hair-conditioning active substances d).

Cationic surfactants (di) which are suitable as contemplated herein are preferably understood to be monomeric cationic or amphoteric ammonium compounds, monomeric amines and/or aminoamides. From this array of possible cationic surfactants (di), the following groups have proven to be particularly suitable and can be used, taken individually, in an amount of from about 0.001 to about 10.0 wt. %. This amount also should not be undershot or exceeded if a mixture of different compounds of the quaternary compounds is used.

As contemplated herein, cationic surfactants of the quaternary ammonium compound type, esterquats and amidoamines are particularly preferred. Preferred quaternary ammonium compounds are ammonium halides, in particular chlorides and bromides, such as alkyltrimethylammonium chlorides, dialkyldimethylammonium chlorides, and trialkylmethylammonium chlorides, for example cetyl trimethyl ammonium chloride, stearyl trimethyl ammonium chloride, distearyl dimethyl ammonium chloride, lauryl dimethyl ammonium chloride, lauryl dimethyl benzyl ammonium chloride, behentrimethyl ammonium chloride, and tricetyl methyl ammonium chloride and the imidazolium compounds known under the INCI names Quaternium-27 and Quaternium-83. The long alkyl chains of the above-mentioned surfactants preferably have 10 to 22 carbon atoms. Further cationic surfactants that can be used as contemplated herein are the quaternised protein hydrolysates, which have already been described in the section discussing protein hydrolysates.

The alkyl amidoamines are usually produced by amidation of natural or synthetic fatty acids and fatty acid cuts with dialkylamino amines and are exemplified especially by their good biodegradability, in addition to a good conditioning effect. A compound from this substance group that is particularly suitable as contemplated herein is the stearamidopropyl dimethylamine commercially obtainable under the name Tegoamid® S 18.

Quaternary ester compounds which are easily biodegradable and which are therefore likewise preferred as contemplated herein are what are known as “esterquats”. Esterquats are known substances that contain both at least one ester function and at least one quaternary ammonium group as structural element. Preferred esterquats are quaternised ester salts of fatty acids with triethanolamine, quaternised ester salts of fatty acids with diethanolalkylamines, and quaternised ester salts of fatty acids with 1,2-dihydroxypropyldialkylamines. Such products are sold for example under the trademarks Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such esterquats.

In particular with the use of a cationic surfactant (di) of the alkyl trimethyl ammonium halide type as described above, it has been found that the nourishing effect of the hair cleansing conditioners as contemplated herein can be further increased, and in particular the stability of the agents can be improved further still.

In a further preferred embodiment the hair cleansing conditioners as contemplated herein therefore additionally contain at least one cationic surfactant (di) of the alkyl trimethyl ammonium halide type, preferably lauryl trimethyl ammonium chloride, cetyl trimethyl ammonium chloride, cetyl trimethyl ammonium bromide, cetyl trimethyl ammonium-methosulfate, dicetyl dimethyl ammonium chloride, tricetyl methyl ammonium chloride, stearyl trimethyl ammonium chloride, distearyl dimethyl ammonium chloride, lauryl dimethyl benzyl ammonium chloride, behenyl trimethyl ammonium chloride, behenyl trimethyl ammonium bromide and/or behenyl trimethyl ammonium methosulfate and in particular the compounds known under the INCI names Cetriumonium Chloride and/or Behentrimonium Chloride.

Suitable oil, wax and/or fat components (dii) can be selected preferably from mineral and/or natural oil components and/or from fatty substances. Triglycerides and mixtures of triglycerides are usually used as natural (plant) oils. Preferred natural oils are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheatgerm oil, pumpkin seed oil, mallow oil, hazelnut oil, safflower oil, canola oil, sasanqua oil, jojoba oil, rambutan oil, cocoa butter and Shea butter. In particular, petroleum oils, paraffin oils and isoparaffin oils and synthetic hydrocarbons are used as mineral oils. An example of a hydrocarbon that can be used is, for example, the commercially available product 1,3-di-(2-ethylhexyl)-cyclohexane (Cetiol® S). A dialkyl ether can also be used as oil component. Dialkyl ethers that can be used are in particular di-n-alkyl ethers having a total of between 12 and 36 carbon atoms, in particular 12 to 24 carbon atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether and also di-tert-butyl ether, di-iso-pentyl ether, di-3-ethyl decyl ether, tert-butyl-n-octyl ether, iso-pentyl-n-octyl ether and 2-methylpentyl-n-octyl ether. The di-n-octyl ether commercially available under the name Cetiol® OE is particularly preferred. Fatty substances are understood to mean fatty acids, fatty alcohols and also natural and synthetic waxes, which can be present either in solid form or in liquid form in aqueous dispersion. Linear and/or branched, saturated and/or unsaturated fatty acids having 6 to 30 carbon atoms can be used as fatty acids. Fatty acids having 10 to 22 carbon atoms are preferred. Examples include the isostearic acids, such as the commercial products Emersol® 871 and Emersol® 875, and isopalmitic acids such as the commercial product Edenor® IP 95, and also all further fatty acids sold under the commercial names Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof. The fatty acid cuts obtainable from coconut oil or palm oil are usually particularly preferred; the use of stearic acid is generally preferred in particular. Saturated, mono- or polyunsaturated, branched or unbranched fatty alcohols having C6 to C30, preferably C10 to C22, and very particularly preferably C12 to C22 carbon atoms can be used as fatty alcohols. For example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, caprin alcohol, linoleyl alcohol, linolenyl alcohol and behenyl alcohol, and the guerbet alcohols thereof can be used, wherein this list is intended to be exemplary and non-limiting. The fatty alcohols, however, preferably originate from natural fatty acids, wherein recovery from the esters of fatty acids by reduction can usually be assumed. Fatty alcohol cuts which are produced by reduction of naturally occurring triglycerides, such as beef tallow, palm oil, peanut oil, rapeseed oil, cottonseed oil, soybean oil, sunflower oil and linseed oil, or fatty acid esters formed from the transesterification products thereof with corresponding alcohols and which therefore represent a mixture of different fatty alcohols can also be used as contemplated herein. Such substances can be purchased for example under the names Stenol®, for example Stenol® 1618, or Lanette®, for example Lanette® 0, or Lorol®, for example Lorol® C8, Lorol® C14, Lorol® C18, Lorol® C8-18, HD-Ocenol®, Crodacol®, for example Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Of course, lanolin alcohols can also be used as contemplated herein, as can be purchased for example under the names Corona®, White Swan®, Coronet® or Fluilan®. Solid paraffins or isoparaffins, carnauba wax, beeswax, candelilla wax, ozokerite, ceresin, spermaceti, sunflower wax, fruit wax, such as apple wax or citrus wax, and microwax from PE or PP can be used as natural or synthetic waxes. Such waxes are obtainable for example from the company Kahl & Co., Trittau. Further fatty substances are, for example

ester oils. Ester oils are understood to be the esters of C6 to C30 fatty acids having C2 to C30 fatty alcohols. The monoesters of fatty acids with alcohols having 2 to 24 carbon atoms are preferred. Examples of used fatty acid components in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof.

Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachyl alcohol, gadoleyl alcohol, behenyl alcohol, erucyl alcohol and brassidyl alcohol and technical mixtures thereof. Particularly preferred are isopropyl myristate (Rilanit® IPM), isononanoic acid C16-C18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V),

Dicarboxylic acid esters such as di-n-butyl adipate, di-(2-ethylhexyl) adipate, di-(2-ethylhexyl) succinate and di-isotridecyl acelaat, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate, and neopentyl glycol dicaprylate,

symmetrical, asymmetrical or cyclic esters of carbonic acid with fatty alcohols,

glycerol carbonate or dicaprylyl carbonate (Cetiol® CC),

ethoxylated or non-ethoxylated mono-, di-, and tri-fatty acid esters of saturated and/or unsaturated linear and/or branched fatty acids with glycerol, such as Monomuls® 90-018, Monomuls® 90-L12, Cetiol® HE or Cutina® MD.

Particularly preferred oil, wax and/or fat components (dii) in the sense of the present disclosure are selected from fatty alcohols and/or plant oils, in particular on account of their nourishing properties and on account of the fact that they do not negatively influence the stability of the compositions as contemplated herein. Examples of very particularly preferred constituents (dii) are cetyl alcohol, stearyl alcohol, cetearyl alcohol and/or one or more of the aforementioned plant oils.

The proportion by weight of the oil, wax and/or fat components (dii) in the total weight of the hair cleansing conditioners as contemplated herein is preferably from about 0.01 to about 5.00 wt. %, particularly preferably from about 0.05 to about 4.00 wt. %, and in particular from about 0.10 to about 3.00 wt. %.

Suitable vitamins (diii) are preferably understood to mean the following vitamins, provitamins and vitamin precursors and derivatives thereof:

Vitamin A: the group of substances referred to as vitamin A includes retinol (vitamin A1) and 3,4-didehydroretinol (vitamin A2). β-carotin is the provitamin of retinol. Examples of suitable vitamin A components include vitamin A acid and esters thereof, vitamin A aldehyde, and vitamin A alcohol as well as esters thereof, such as the palmitate and acetate.

Vitamin B: the vitamin B group or the vitamin B complex includes, inter alia,
vitamin B1 (thiamin)
vitamin B2 (riboflavin)
vitamin B3. This often includes the compounds of nicotinic acid and nicotinic acid amide (niacinamide).
vitamin B5 (pantothenic acid and panthenol). In the context of this group, panthenol is preferably used. Useable derivatives of panthenol are especially the esters and ethers of panthenol, pantolactone, and also cationically derivatised panthenols. Specific representatives are, for example, panthenol triacetate, panthenol monoethyl ether and monoacetate thereof, as well as cationic panthenol derivatives.
vitamin B6 (pyridoxine and also pyridoxamine and pyridoxal).

Vitamin C (ascorbic acid): the use in the form of the palmitic acid ester, the glucosides, or phosphates can be preferred. The use in combination with tocopherols can also be preferred.

Vitamin E (tocopherols, in particular α-tocopherol).

Vitamin F: the term “vitamin F” is usually understood to mean essential fatty acids, in particular linoleic acid, linolenic acid and arachidonic acid.

Vitamin H: The compound (3aS,4S, 6aR)-2-oxohexahydrothienol[3,4-d]-imidazol-4-valeric acid denotes vitamin H, for which the trivial name however (biotin) has become accepted.

Vitamins, pro-vitamins and vitamin precursors (diii) from groups A, B, E and H are particularly preferred. Nicotinic acid amide, biotin, pantolactone and/or panthenol are very particularly preferred. The proportion by weight of the vitamin(s), vitamin derivative(s) and/or vitamin precursor(s) (diii) in the total weight of the hair cleansing conditioners as contemplated herein is preferably from about 0.001 to about 2.00 wt. %, particularly preferably from about 0.005 to about 1.00 wt. %, and in particular from about 0.01 to about 0.50 wt. %.

Further active substances, auxiliaries and additives which can be contained with preference in the hair cleansing conditioners according to the example include, for example:

plant extracts,

humectants,

perfumes,

UV filters,

thickening agents such as gelatins or plant gums, for example agar-agar, guar gum, alginates, xanthan gum, gum arabic, karaya gum, locust bean gum, linseed gums, dextrans, cellulose derivatives, for example methyl cellulose, hydroxyalkyl cellulose and carboxymethyl cellulose, starch fractions and derivatives such as amylose, amylopectin and dextrins, clays and sheet silicates, such as bentonite or fully synthetic hydrocolloids, such as polyvinyl alcohol, the Ca, Mg or Zn soaps,

structuring agents such as maleic acid and lactic acid,

dimethyl isosorbide,

cyclodextrins,

active substances that improve the fibre structure, in particular mono-, di- and oligosaccharides, such as glucose, galactose, fructose, fruit sugar and lactose,

dyes to colour the agent,

substances to adjust the pH value, such as α- and β-hydroxycarboxylic acids, such as citric acid, lactic acid, malic acid, glycolic acid,

active substances such as bisabolol and/or allantoin,

complexing agents, such as EDTA, NTA, β-alanine diacetic acid and phosphonic acids,

ceramides. Ceramides are understood to be N-acyl sphingosine (fatty acid amides of sphingosine) or synthetic analogues of such lipids (known as pseudoceramides),

antioxidants,

preservatives, such as sodium benzoate or salicylic acid,

additional viscosity adjusters such as salts (NaCl).

A second subject as contemplated herein is the cosmetic use of the hair cleansing conditioner as contemplated herein for the mild cleansing and excellent nourishment of hair, in particular of brittle, damaged, dull, thin and/or lank hair.

A third subject as contemplated herein is a method for treating hair, in which the hair cleansing conditioner as contemplated herein is applied to the hair, which is preferably wet, is massaged in, and is rinsed out after a reaction time.

A method as contemplated herein forming the third subject as contemplated herein in which the hair cleansing conditioner is applied to the hair as a cleansing nourishing aerosol mousse from an aerosol container is particularly preferred.

That which has been said in relation to the hair cleansing conditioners as contemplated herein applies, mutatis mutandis, for preferred embodiments of the use as contemplated herein and of the method as contemplated herein

The following examples are intended to explain preferred embodiments as contemplated herein, but without limiting the present disclosure thereto.

### EXAMPLES

### 1. Composition of Hair Cleansing Conditioners as Contemplated Herein

The following hair cleansing conditioners as contemplated herein were produced, wherein all numerical values in the following examples—unless otherwise specified—correspond to the amount of the active substance in question in wt. %, in relation to the total weight of the agent:

| | Formula 1 | Formula 2 | Formula 3 | Formula 4 |
|---|---|---|---|---|
| Poly(3-acrylamidopropyl)- | 0.0001-10 | 0.001 to 5.0 | 0.005-3.0 | 0.01-2.0 |
| trimethyl ammonium chloride* | | | | |
| Non-ionic surfactant (bi), | 0.01-10 | 0.10-7.50 | 0.50-6.00 | 1.00-5.00 |
| preferably alkyl(oligo)glycoside, | | | | |
| in particular Lauryl Glucoside, | | | | |
| Coco Glucoside and/or Decyl | | | | |
| Glucoside | | | | |
| Water and any possible further | to 100 | to 100 | to 100 | to 100 |
| auxiliaries and additives | | | | |
| | Formula 5 | Formula 6 | Formula 7 | Formula 8 |
| Poly(3-acrylamidopropyl)- | 0.0001-10 | 0.001 to 5.0 | 0.005-3.0 | 0.01-2.0 |
| trimethyl ammonium chloride* | | | | |
| Non-ionic surfactant (bi), | 0.01-10 | 0.10-7.50 | 0.50-6.00 | 1.00-5.00 |
| preferably alkyl(oligo)glycoside, | | | | |
| in particular Lauryl Glucoside, | | | | |
| Coco Glucoside and/or Decyl | | | | |
| Glucoside | | | | |
| Protein hydrolysate, in particular | 0.01-5.00 | 0.05-4.00 | 0.10-3.00 | 0.10-2.00 |
| Hydrolyzed Keratin | | | | |
| Water and any possible further | to 100 | to 100 | to 100 | to 100 |
| auxiliaries and additives | | | | |
| | Formula 9 | Formula 10 | Formula 11 | Formula 12 |
| Poly(3-acrylamidopropyl)- | 0.0001-10 | 0.001 to 5.0 | 0.005-3.0 | 0.01-2.0 |
| trimethyl ammonium chloride* | | | | |
| Non-ionic surfactant (bi), | 0.01-10 | 0.10-7.50 | 0.50-6.00 | 1.00-5.00 |
| preferably alkyl(oligo)glycoside, | | | | |
| in particular Lauryl Glucoside, | | | | |
| Coco Glucoside and/or Decyl | | | | |
| Glucoside | | | | |
| Propellant, | 1.00-50 | 2.00-40 | 3.00-30 | 4.00-20 |
| in particular propane/butane | | | | |
| Water and any possible further | to 100 | to 100 | to 100 | to 100 |
| auxiliaries and additives | | | | |
| | Formula | | | |
| | 13 | Formula 14 | Formula 15 | Formula 16 |
| Poly(3-acrylamidopropyl)- | 0.0001-10 | 0.001 to 5.0 | 0.005-3.0 | 0.01-2.0 |
| trimethyl ammonium chloride* | | | | |
| Non-ionic surfactant (bi), | 0.01-10 | 0.10-7.50 | 0.50-6.00 | 1.00-5.00 |
| preferably alkyl(oligo)glycoside, | | | | |
| in particular Lauryl Glucoside, | | | | |
| Coco Glucoside and/or Decyl | | | | |
| Glucoside | | | | |
| Propellant, | 1.00-50 | 2.00-40 | 3.00-30 | 4.00-20 |
| in particular propane/butane | | | | |
| Protein hydrolysate, in particular | 0.01-5.00 | 0.05-4.00 | 0.10-3.00 | 0.10-2.00 |
| Hydrolyzed Keratin | | | | |
| Water and any possible further | to 100 | to 100 | to 100 | to 100 |
| auxiliaries and additives | | | | |
| | Formula | | | |
| | 17 | Formula 18 | Formula 19 | Formula 20 |
| Poly(3-acrylamidopropyl)- | 0.0001-10 | 0.001 to 5.0 | 0.005-3.0 | 0.01-2.0 |
| trimethyl ammonium chloride* | | | | |
| Amphoteric surfactant (bii), | 0.10-15.00 | 0.50-12.50 | 1.00-10.00 | 1.50-7.50 |
| preferably Cocoampho(di)acetate | | | | |
| and/or Cocamidopropyl betaine | | | | |
| Water and any possible further | to 100 | to 100 | to 100 | to 100 |
| auxiliaries and additives | | | | |
| | Formula | | | |
| | 21 | Formula 22 | Formula 23 | Formula 24 |
| Poly(3-acrylamidopropyl)- | 0.0001-10 | 0.001 to 5.0 | 0.005-3.0 | 0.01-2.0 |
| trimethyl ammonium chloride* | | | | |
| Amphoteric surfactant (bii), | 0.10-15.00 | 0.50-12.50 | 1.00-10.00 | 1.50-7.50 |
| preferably Cocoampho(di)acetate | | | | |
| and/or Cocamidopropyl betaine | | | | |
| Protein hydrolysate, in particular | 0.01-5.00 | 0.05-4.00 | 0.10-3.00 | 0.10-2.00 |
| Hydrolyzed Keratin | | | | |
| Water and any possible further | to 100 | to 100 | to 100 | to 100 |
| auxiliaries and additives | | | | |
| | Formula | | | |
| | 25 | Formula 26 | Formula 27 | Formula 28 |
| Poly(3-acrylamidopropyl)- | 0.0001-10 | 0.001 to 5.0 | 0.005-3.0 | 0.01-2.0 |
| trimethyl ammonium chloride* | | | | |
| Amphoteric surfactant (bii), | 0.10-15.00 | 0.50-12.50 | 1.00-10.00 | 1.50-7.50 |
| preferably Cocoampho(di)acetate | | | | |
| and/or Cocamidopropyl betaine | | | | |
| Propellant, | 1.00-50 | 2.00-40 | 3.00-30 | 4.00-20 |
| in particular propane/butane | | | | |
| Water and any possible further | to 100 | to 100 | to 100 | to 100 |
| auxiliaries and additives | | | | |
| | Formula | | | |
| | 29 | Formula 30 | Formula 31 | Formula 32 |
| Poly(3-acrylamidopropyl)- | 0.0001-10 | 0.001 to 5.0 | 0.005-3.0 | 0.01-2.0 |
| trimethyl ammonium chloride* | | | | |
| Amphoteric surfactant (bii), | 0.10-15.00 | 0.50-12.50 | 1.00-10.00 | 1.50-7.50 |
| preferably Cocoampho(di)acetate | | | | |
| and/or Cocamidopropyl betaine | | | | |
| Propellant, | 1.00-50 | 2.00-40 | 3.00-30 | 4.00-20 |
| in particular propane/butane | | | | |
| Protein hydrolysate, in particular | 0.01-5.00 | 0.05-4.00 | 0.10-3.00 | 0.10-2.00 |
| Hydrolyzed Keratin | | | | |
| Water and any possible further | to 100 | to 100 | to 100 | to 100 |
| auxiliaries and additives | | | | |
| | Formula | | | |
| | 33 | Formula 34 | Formula 35 | Formula 36 |
| Poly(3-acrylamidopropyl)- | 0.0001-10 | 0.001 to 5.0 | 0.005-3.0 | 0.01-2.0 |
| trimethyl ammonium chloride* | | | | |
| Non-ionic surfactant (bi), | 0.01-10 | 0.10-7.50 | 0.50-6.00 | 1.00-5.00 |
| preferably alkyl(oligo)glycoside, | | | | |
| in particular Lauryl Glucoside, | | | | |
| Coco Glucoside and/or Decyl | | | | |
| Glucoside and/or | 0.10-15.00 | 0.50-12.50 | 1.00-10.00 | 1.50-7.50 |
| amphoteric surfactant (bii), | | | | |
| preferably Cocoampho(di)acetate | | | | |
| and/or Cocamidopropyl betaine | | | | |
| Propellant, | 1.00-50 | 2.00-40 | 3.00-30 | 4.00-20 |
| in particular propane/butane | | | | |
| Protein hydrolysate, in particular | 0.01-5.00 | 0.05-4.00 | 0.10-3.00 | 0.10-2.00 |
| Hydrolyzed Keratin | | | | |
| Cationic surfactant (di), in | 0.001-10.0 | 0.01-7.50 | 0.05-6.00 | 0.10-5.00 |
| particular Cetrimonium Chloride | | | | |
| and/or Behentrimonium Chloride | | | | |
| Water and any possible further | to 100 | to 100 | to 100 | to 100 |
| auxiliaries and additives | | | | |
| | Formula | | | |
| | 37 | Formula 38 | Formula 39 | Formula 40 |
| Poly(3-acrylamidopropyl)- | 0.0001-10 | 0.001 to 5.0 | 0.005-3.0 | 0.01-2.0 |
| trimethyl ammonium chloride* | | | | |
| Non-ionic surfactant (bi), | 0.01-10 | 0.10-7.50 | 0.50-6.00 | 1.00-5.00 |
| preferably alkyl(oligo)glycoside, | | | | |
| in particular Lauryl Glucoside, | | | | |
| Coco Glucoside and/or Decyl | | | | |
| Glucoside and/or | 0.10-15.00 | 0.50-12.50 | 1.00-10.00 | 1.50-7.50 |
| amphoteric surfactant (bii), | | | | |
| preferably Cocoampho(di)acetate | | | | |
| and/or Cocamidopropyl betaine | | | | |
| Propellant, | 1.00-50 | 2.00-40 | 3.00-30 | 4.00-20 |
| in particular propane/butane | | | | |
| Protein hydrolysate, in particular | 0.01-5.00 | 0.05-4.00 | 0.10-3.00 | 0.10-2.00 |
| Hydrolyzed Keratin | | | | |
| Vitamin (diii), in particular | 0.001-2.00 | 0.005-1.50 | 0.01-1.00 | 0.05-0.75 |
| Panthenol and/or Niacinamide | | | | |
| Water and any possible further | to 100 | to 100 | to 100 | to 100 |
| auxiliaries and additives | | | | |

| | 41 | 42 | 43 | 44 |
|---|---|---|---|---|
| Poly(3-acrylamidopropyl)- | 0.3 | 0.5 | 0.3 | 0.5 |
| trimethyl ammonium chloride* | | | | |
| Lauryl Glucoside | 1.3 | | 1.3 | |
| Sodium Cocoamphoacetate | | 2.0 | | 2.0 |
| Cocamide MEA | | 2.0 | | 2.0 |
| Hydrolyzed Keratin | 0.2 | 0.2 | 0.2 | 0.2 |
| Behentrimonium Chloride | 3.0 | 0.3 | 3.0 | 0.3 |
| Panthenol | 0.2 | | 0.2 | |
| Stearyl Alcohol | | 1.8 | | 1.8 |
| Perfume | 0.6 | 0.7 | 0.6 | 0.7 |
| Lactic Acid | 0.8 | 0.4 | 0.8 | 0.4 |
| Propane/Butane | | | 8.0 | 9.0 |
| Preservative(s) | q.s. | q.s. | q.s. | q.s. |
| Water | to 100 | to 100 | to 100 | to 100 |

*Used trade product: N-DurHance A-100 from Ashland Inc.

### 2. Assessment of Hair Cleansing Conditioners as Contemplated Herein

100% healthy women (no allergies or skin problems) between 18 and 50 years old (50% 18-34 years; 50% 35-50 years) were surveyed, who had tested the hair cleansing conditioners as contemplated herein in comparison with competition and/or comparison products (in anonymised and coded form) in a blind test between 29 Oct. and 26 Nov. 2015. Half of the women had used the product as contemplated herein 3-4 times per week, and the other half had used the comparison product.

### 2.1 Assessment of Skin Compatibility

The skin compatibility of the hair cleansing conditioner as contemplated herein according to formula 41 was evaluated compared to a conventional nourishing shampoo (comprising, instead of Lauryl Glucoside and Behentrimonium Chloride, 7 wt. % (active substance) sodium lauryl ether sulphate, and comprising, instead of N-DurHance A-1000, 0.3 wt. % Polyquaternium-10) with the aid of a dermatological patch test. The results of the patch test can be seen in the following Tables 1 and 2:

TABLE 1
a) Treatment with the cleansing conditioner according to formula 41

| | | Reddening | | | | | | | | Dandruff | | | | | | | |
|---|---|---|---|---|---|---|---|---|---|---|---|---|---|---|---|---|---|
| Time | | (erythema) | | | | Oedema | | | | formation | | | | Cracking | | | |
| Intensity | No reaction | 1 | 2 | 3 | 4 | 1 | 2 | 3 | 4 | 1 | 2 | 3 | 4 | 1 | 2 | 3 | 4 |
| 6 hours | 14/20 | 5 | 1 | 0 | 0 | 0 | 0 | 0 | 0 | 0 | 0 | 0 | 0 | 0 | 0 | 0 | 0 |
| 24 hours | 17/20 | 3 | 0 | 0 | 0 | 0 | 0 | 0 | 0 | 0 | 0 | 0 | 0 | 0 | 0 | 0 | 0 |
| 48 hours | 18/20 | 1 | 0 | 0 | 0 | 0 | 0 | 0 | 0 | 1 | 0 | 0 | 0 | 0 | 0 | 0 | 0 |
| 72 hours | 19/20 | 0 | 0 | 0 | 0 | 0 | 0 | 0 | 0 | 1 | 0 | 0 | 0 | 0 | 0 | 0 | 0 |

TABLE 2
b) Treatment with the aforementioned comparison nourishing shampoo

| | | Reddening | | | | | | | | Dandruff | | | | | | | |
|---|---|---|---|---|---|---|---|---|---|---|---|---|---|---|---|---|---|
| Time | | (erythema) | | | | Oedema | | | | formation | | | | Cracking | | | |
| Intensity | No reaction | 1 | 2 | 3 | 4 | 1 | 2 | 3 | 4 | 1 | 2 | 3 | 4 | 1 | 2 | 3 | 4 |
| 6 hours | 8/20 | 6 | 6 | 0 | 0 | 0 | 0 | 0 | 0 | 0 | 0 | 0 | 0 | 0 | 0 | 0 | 0 |
| 24 hours | 5/20 | 6 | 7 | 2 | 0 | 0 | 0 | 0 | 0 | 0 | 0 | 0 | 0 | 0 | 0 | 0 | 0 |
| 48 hours | 5/20 | 9 | 4 | 0 | 0 | 0 | 0 | 0 | 0 | 1 | 1 | 0 | 0 | 0 | 0 | 0 | 0 |
| 72 hours | 7/20 | 6 | 0 | 0 | 0 | 0 | 0 | 0 | 0 | 7 | 0 | 0 | 0 | 0 | 0 | 0 | 0 |

It can be seen from the values in Tables 1 and 2 that the hair cleansing conditioners as contemplated herein have an improved skin compatibility compared to a conventional nourishing shampoo based on anionic sulphate surfactants.

### 2.2 Assessment of the Nourishing Properties

In a second test, the hair cleansing conditioner as contemplated herein according to formula 41 was tested by users (see above) with a commercial product (Co-Wash Cleansing Conditioner) in a blind test. The composition of the Co-Wash conditioner corresponded approximately to the compositions of document U.S. Pat. No. 6,723,309 B1, which was already noted at an earlier point in this application (composition of the Co-Wash conditioner according to INCI: Water, Stearyl Alcohol, Cetyl Alcohol, Stearamidopropyl Dimethylamine, Glutamic Acid, Bis-Aminopropyl Dimethicone, Fragrance, Benzyl Alcohol, EDTA, Citric Acid, Sodium Chloride, Panthenol, Panthenyl Ethyl Ether, Simmondsia Chinensis (Jojoba) Seed Oil, Cocos Nucifera (Coconut) Oil, Methylchloroisothiazolinone, Methylisothiazolinone).

The results of the survey after use of the products can be found in the following table:

| | Hair cleansing | |
|---|---|---|
| | conditioner | Co-Wash |
| | according to | Cleansing |
| | formula 41 | Conditioner |
| No weighing-down of the hair | 91 | 79 |
| Gives the treated hair volume and fullness | 90 | 72 |
| Gives the hair resilience and elasticity | 89 | 75 |
| Good styling properties | 88 | 75 |
| Good combability when wet | 63 | 51 |
| Good feel of the wet hair | 62 | 51 |
| Good properties with regard to rinsing out | 57 | 51 |
| Good hair feel during the rinse-out process | 66 | 49 |
| Good combability when dry | 66 | 47 |
| Good feel of the dry hair | 61 | 55 |
| Improved hair shine | 65 | 52 |
| Improved softness | 67 | 54 |
| Mild on the scalp | 70 | 56 |
| Healthy appearance of the hair | 68 | 56 |

The results in the table show that the compositions as contemplated herein are slightly to significantly superior to the conventional hair cleansing conditioners for all points.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair cleansing conditioner comprising—in relation to the weight of the total composition—
a) from about 0.0001 to about 10 wt. % of a cationic 3-acrylamidopropyl trimethylammonium salt homopolymer, and
b) from about 0.01 to about 20 wt. % of at least one non-ionic surfactant and/or at least one amphoteric surfactant.

2. A hair cleansing conditioner according to claim 1, wherein the 3-acrylamidopropyl trimethylammonium salt homopolymer a) is selected from (3-acrylamidopropyltrimethylammonium chloride homopolymers.

3. A hair cleansing conditioner according to claim 1, comprising at least one non-ionic surfactant (bi).

4. A hair cleansing conditioner according to claim 3, comprising—in relation to its weight—from about 0.01 to about 10 wt. % of the at least one non-ionic surfactant (bi).

5. A hair cleansing conditioner according to claim 1, comprising at least one amphoteric surfactant (bii).

6. A hair cleansing conditioner according to claim 5, comprising—in relation to its weight—from about 0.10 to about 15.00 wt.

7. A hair cleansing conditioner according to claim 1, substantially free from anionic surfactants.

8. A hair cleansing conditioner according to claim 1, which is present in the form of an aerosol mousse in an aerosol container and additionally comprises at least one propellant in a proportion by weight of from about 1.00 to about 50 wt. % in the total weight of the composition.

9. A hair cleansing conditioner according to claim 1, comprising at least one protein hydrolysate in a proportion by weight of from about 0.01 to about 5.00 wt. % in the total weight of the composition.

10. A hair cleansing conditioner according to claim 1, comprising
a. from about 0.0001 to about 10 wt. % of a cationic 3-acrylamidopropyl trimethylammonium salt homopolymer,
b. from about 0.01 to about 10 wt. % of at least one non-ionic surfactant (bi), and
c. from about 1.00 to about 50 wt. % of at least one propellant, and/or
d. from about 0.01 to about 5.00 wt. % of at least one protein hydrolysate,
wherein the specified values relate to the total weight of the hair cleansing conditioner.

11. A hair cleansing conditioner according to claim 1, comprising
a) from about 0.0001 to about 10 wt. % of a cationic 3-acrylamidopropyl trimethylammonium salt homopolymer,
b) from about 0.10 to about 15 wt. % of at least one amphoteric surfactant (bii),
c) from about 1.00 to about 50 wt. % of at least one propellant gas, and/or
from about 0.01 to about 5.00 wt. % of at least one protein hydrolysate,
wherein the specified values relate to the total weight of the hair cleansing conditioner.

12. (canceled)

13. A method for treating hair, in which a hair cleansing conditioner is applied to the hair, is massaged in, and is rinsed out after a reaction time, wherein the hair cleansing conditioner comprises—in relation to the weight of the total composition—
a) from about 0.0001 to about 10 wt. % of a cationic 3-acrylamidopropyl trimethylammonium salt homopolymer, and
b) from about 0.01 to about 20 wt. % of at least one non-ionic surfactant and/or at least one amphoteric surfactant.

14. (canceled)

15. A hair cleansing conditioner according to claim 2, wherein the at least one non-ionic surfactant (bi) is selected from alkyl(oligo)glycosides of general formula:
RO-[G]x
wherein R stands for an alkyl group and/or alkenyl group having from about 4 to about 22 C atoms, G stands for a sugar group having from about 5 or about 6 C atoms, and x stands for the numbers from about 1 to about 10.

16. A hair cleansing conditioner according to claim 4, comprising—in relation to its weight—from about 1.00 to about 5.00 wt. %, of an alkyl(oligo)glycoside.

17. A hair cleansing conditioner according to claim 5, wherein the at least one amphoteric surfactant (bii) is selected from at least one of the compounds known under the INCA names Sodium Cocoamphoacetate, Disodium Cocoamphodiacetate, Sodium Lauroamphoacetate, Sodium Lauroamphodiacetate, Sodium Cocoamphopropionate, Disodium Cocoamphodipropionate, Coco Betaine, Lauryl Betaine, Cocamidopropyl Betaine and/or Lauramidopropyl Betaine.

18. A hair cleansing conditioner according to claim 6, comprising—in relation to its weight—from about 1.50 to about 7.50 wt. %, of at least one of the compounds known under the INCI names Sodium Cocoampho(di)acetate and/or Cocamidopropyl Betaine.

19. A hair cleansing conditioner according to claim 1, substantially free from anionic sulphate surfactants.

20. A hair cleansing conditioner according to claim 8, wherein the at least one propellant is present in a proportion by weight of from about 4.00 to about 20.0 wt. %, in the total weight of the composition.

21. A hair cleansing conditioner according to claim 1, comprising
a. from about 0.0001 to about 10 wt. % of a (3-acrylamidopropyl)trimethylammonium chloride homopolymer,
b. from about 0.01 to about 10 wt. % of at least one alkyl(oligo)glycoside),
c. from about 1.00 to about 50 wt. % of at least one propellant, and/or
d. from about 0.01 to about 5.00 wt. % of at least one protein hydrolysate known under the INCI name Hydrolyzed Keratin,
wherein the specified values relate to the total weight of the hair cleansing conditioner.

22. A hair cleansing conditioner according to claim 1, comprising
a) from about 0.0001 to about 10 wt. % of a (3-acrylamidopropyl)trimethylammonium chloride homopolymer,
b) from about 0.10 to about 15 wt. % of at least one of the compounds known under the INCI names Sodium Cocoampho(di)acetate and/or Cocoamidopropyl Betaine,
c) from about 1.00 to about 50 wt. % of at least one propellant gas, and/or
from about 0.01 to about 5.00 wt. % of at least one protein hydrolysate known under the INCI name Hydrolyzed Keratin,
wherein the specified values relate to the total weight of the hair cleansing conditioner.
